(19) United States
(12) Patent Application Publication
(10) Pub. No.: US 2026/0360530 A1
(43) Pub. Date: Nov. 12, 2026

(54) VENDOR-AGNOSTIC REMOTE-CONTROLLED SCREEN OVERLAY FOR COLLABORATION IN A VIRTUALIZED RADIOLOGY ENVIRONMENT

(71) Applicant: KONINKLIJKE PHILIPS N.V., EINDHOVEN (NL)

(72) Inventor: THOMAS ERIK AMTHOR, HAMBURG (DE)

(21) Appl. No.: 19/100,340

(22) PCT Filed: Jul. 28, 2023
(86) PCT No.: PCT/EP2023/071055

§ 371 Date: Jan. 31, 2025

Related U.S. Application Data
(60) Provisional application No. 63/394,988, filed on Aug. 4, 2022.

(30) Foreign Application Priority Data
Aug. 31, 2022 (EP) 22193088.6

(51) Int. Cl. G16H 40/67; G06F 3/14; G16H 30/40; G16H 80/00; H04N 21/431; H04N 21/4788

(52) U.S. Cl. CPC G16H 40/67; G06F 3/1454; G16H 30/40; G16H 80/00; H04N 21/4312; H04N 21/4788

(57) ABSTRACT

A remote expert assists a local operator of a medical imaging device during a medical imaging examination. A user interface (UI) is provided on an assistor electronic device operable by the remote expert. The UI displays controller video output by an electronic controller of the medical imaging device. Graphical annotations for updating the controller video are received via at least one user input device of the assistor electronic device. The controller video overlaid with the graphical annotations is displayed on a controller display of the medical imaging device. In one approach, this entails splitting the controller video into a plurality of feeds, transmitting one of the feeds to the assistor electronic device, and transmitting another of the feeds to a screen overlay processor that also receives the graphical annotations from the assistor electronic device and that generates the controller video overlaid with the graphical annotations.

The following relates generally to the imaging arts, remote imaging assistance arts, remote imaging examination monitoring arts, and related arts.

### BACKGROUND OF THE INVENTION

Medical imaging, such as computed tomography (CT) imaging, magnetic resonance imaging (MRI), positron emission tomography (PET) imaging, fluoroscopy imaging, and so forth, is a critical component of providing medical care, and is used in a wide range of medical fields, such as cardiology, oncology, neurology, orthopedics, to name a few. The operator of the medical imaging device used to acquire the medical images is typically a trained technologist, while interpretation of the medical images is often handled by a medical specialist such as a radiologist. Interpretation of radiology reports or findings by the radiologist can be handled by the patient's general practitioner (GP) physician or a medical specialist such as a cardiologist, oncologist, orthopedic surgeon, or so forth.

Currently, medical imaging is in high demand. As the world population ages, the demand for quick, safe, high quality medical imaging will only continue to grow, putting further pressure on imaging centers and their staff. Under such conditions, errors can occur, and can often be costly. One approach for imaging centers to boost efficiency and grow operations at no extra labor costs is through a radiology operations command center (ROCC) system. Radiology operations command centers enable teams to work across the entire network of imaging sites, providing their expertise as needed and remotely assisting less experienced technologists in carrying out high quality scans. Remote technologists or experts can monitor the local operators of scanning procedures through cameras installed in the scanning areas (or from other sources, such as sensors (including radar sensors), console video feeds, microphones connected to Internet of Things (IoT) devices, and so forth. In addition, these sources can be supplemented by other data sources like Health-Level 7 (HL7), Digital Imaging and Communications in Medicine (DICOM), Electronic Health Record (EHR) databases, and so forth.

When collaborating via the ROCC platform, the local and remote technologists can communicate via an audio or video call on a tablet computer. In addition, the remote technologist can see the local console screen via video capture and transmission. Hence, the remote technologist can observe control or other actions taken by the local technologist, and can also observe the mouse pointer or other on-display user interfacing elements controlled by the local technologist.

In many situations, due to safety or security regulations, the remote technologist will only be allowed to view the local screen but not interact with the scanner system or local input device. Moreover, explanations of user interface (UI) interactions or identification of specific areas in an image must be conducted via audio communication, as the remote operator has no way of interacting with the local console screen. This makes assistive sessions with unexperienced local staff particularly difficult.

The following discloses certain improvements to overcome these problems and others.

### SUMMARY OF THE INVENTION

In one aspect, an assistive system is disclosed for providing remote assistance to a local operator of a medical imaging device that has an electronic imaging device controller and a controller display. The assistive system includes an assistor electronic device including an electronic processor, an assistor display, and at least one user input device. The assistive system further includes a video router configured to: receive controller video from the electronic imaging device controller; receive overlay content or video from the assistor electronic device; output the controller video to the assistor electronic device; and output annotated video to the controller display, whereby the annotated video is displayed on the controller display. The assistor electronic device is programmed to provide a graphical user interface (GUI) operative to display the controller video on the assistor display, and receive graphical annotations of the controller video from the at least one user input device. The assistor electronic device is further programmed to generate the overlay content or video comprising at least the graphical annotations. The annotated video comprises the controller video overlaid with the graphical annotations.

In another aspect, a method is disclosed of providing assistance from a remote expert to a local operator of a medical imaging device during a medical imaging examination. The method includes: providing a user interface (UI) on an assistor electronic device operable by the remote expert. The UI displays controller video output by an electronic controller of the medical imaging device. The method further includes receiving, via at least one user input device of the assistor electronic device, one or more graphical annotations to update the controller video. The controller video overlaid with the graphical annotations is displayed on a controller display of the medical imaging device.

In another aspect, an assistive system is disclosed for providing remote assistance to a local operator of a medical imaging device having an electronic imaging device controller and a controller display. The assistive system includes an assistor electronic device and a video router. The assistor electronic device includes an electronic processor, an assistor display, and at least one user input device. The video router is configured to: receive controller video from the electronic imaging device controller; receive graphical annotations from the assistor electronic device; output the controller video to the assistor electronic device; and output annotated video to the controller display, whereby the annotated video is displayed on the controller display. The assistor electronic device is programmed to: provide a GUI operative to display the controller video on the assistor display; and receive the graphical annotations of the controller video from the at least one user input device. The annotated video comprises the controller video overlaid with the graphical annotations.

One advantage resides in providing a remote expert or radiologist assisting a technologist in conducting a medical imaging examination with a capability to annotate a screen operable by the technologist.

Another advantage resides in remote expert or radiologist assisting a technologist in conducting a medical imaging examination with a capability to annotate a screen operable by the technologist by without interfering with the local imaging system or host computer.

Another advantage resides in creating screen overlays generated by the remote expert or radiologist directly on the video signal of the local console monitor.

Another advantage resides in creating such screen overlays controlled by a remote operator in real time.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 shows another aspect of the apparatus of FIG. 1.

FIG. 3 shows an example flow chart of operations suitably performed by the apparatus of FIG. 1.

FIG. 4 diagrammatically shows some examples of highlighting overlay elements.

FIGS. 5, 6, and 7 show alternative embodiments to the embodiment shown in FIG. 2.

### DETAILED DESCRIPTION OF EMBODIMENTS

In some current implementations of a Remote Operations Control Center (ROCC), the remote expert receives a scraped copy of the controller screen, and thus can see what a local operator sees. The scraped controller screen viewed by the remote expert typically includes a mouse pointer or other user interfacing element(s) controlled by the local operator, so the local operator may be able to point out features on the screen during communications with the remote expert. However, there is no mechanism by which the remote expert can point to features on the controller screen in a way that the local operator can see.

Embodiments disclosed herein remedy this deficiency by providing a mechanism for the remote expert to draw annotations on the controller display that are visible to the local operator as an overlay on the controller display. To enable this to be vendor-agnostic, in some embodiments the approach operates on the video signal, intercepting it as it passes from the imaging device controller to the display viewed by the local operator and modifying the video signal to add the overlay.

Some existing ROCC implementations employ a Keyboard, Video, & Mouse (KVM) switch to act as a transmitter of the video signal, and a video splitter is configured to split the video signal to two paths: the path to the video display seen by the local operator, and a second path providing the scraped screen that is sent to the remote expert. To implement the overlay disclosed herein, an additional screen overlay processor is interposed in the path from the KVM switch (or more generally, video router) to the local operator display. The operation of the screen overlay processor depends on the nature of the video signal. For a digital video signal such as HMDI, DisplayPort, or digital DVI, the screen overlay processor can be implemented as a digital signal processing (DSP) component, e.g. using a graphical processing unit (GPU) and hardware acceleration to provide sufficient speed in some embodiments. On the other hand, if the video signal is analog, such as an analog DVI signal, then an additional analog-to-digital (A/D) frontend and digital-to-analog (D/A) backend is suitably employed, with DSP being performed on the digitized display content output by the A/D frontend prior to the subsequent D/A backend. In some embodiments, the annotated overlay elements may be received from the remote expert as vectorized or symbolic graphical elements, e.g. represented as lines or shapes delineated by coordinates, along with color and transparency values. The DSP then converts these to a screen overlay that is fused with the received controller display video.

At the remote expert workstation, suitable hardware and software is added to implement the screen annotation user interface (UI). For example, the workstation can have a touch-sensitive display enabling the remote expert to draw on the window presenting the controller display, and/or a mouse pointer, touchpad, or the like can be used for this purpose. As noted, the remote expert workstation in some embodiments converts these annotations to the vectorized or symbolic graphical elements that are sent via the Internet to the screen overlay processor.

One potential concern is that the annotations added by the remote expert could confuse or distract the local operator. To address this potential concern, in some embodiments a “kill switch” is provided at the local operator side that can enable the local operator to turn off the annotations overlay in the interest of safety. As another contemplated variant, additional annotations could be overlaid on the local operator screen beyond the annotations directed created by the remote expert, such as a red box around the entire controller display indicating the remote operator is viewing it.

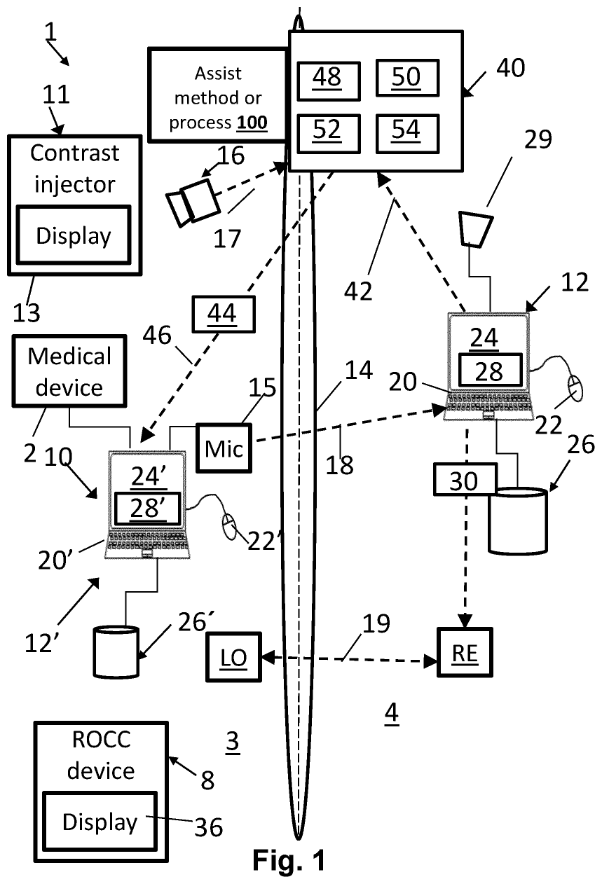

With reference to FIG. 1, an apparatus 1 for providing assistance from a remote medical imaging expert RE (or supertech) to a local technologist operator LO is shown. As shown in FIG. 1, the local operator LO, who operates a medical imaging device (also referred to as an image acquisition device, imaging device, and so forth) 2, is located in a medical imaging device bay 3, and the remote expert RE is disposed in a remote service location or center 4. It should be noted that the “remote expert” RE may not necessarily directly operate the medical imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the medical imaging device bay 3 (this may, for example, in the case of a “remote operator or expert” RE who is a radiologist tasked with peri-examination image review), but more typically the remote service center 4 and the medical imaging device bay 3 are in different buildings, and indeed may be located in different cities, different countries, and/or different continents. In general, the remote location 4 is remote from the imaging device bay 3 in the sense that the remote expert RE cannot directly visually observe the imaging device 2 in the imaging device bay 3 (hence optionally providing a video feed as described further herein).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities. Moreover, the remote service center 4 may provide service to multiple hospitals. The local operator controls the medical imaging device 2 via an electronic imaging device controller 10. The remote operator is stationed at an assistance electronic device 12 (or, more generally, a remote workstation 12 or an electronic controller 12).

To provide for contrast-enhanced imaging in certain imaging modalities, an optional contrast injector 11 is configured to inject the patient with a contrast agent. The contrast injector 11 is a configurable automated contrast injector having a display 13. The user (usually the imaging technologist) loads a vial or syringe of contrast agent (or two, or more, vials of different contrast agent components) into the contrast injector 11, and configures the contrast injector 11 by entering contrast injector settings such as flow rates, volumes, time delays, injection time durations, and/or so forth via a user interface (UI) of the contrast injector 11. The UI may be a touch-sensitive overlay of the display 13, and/or physical buttons, keypad, and/or so forth. In a variant embodiment, the optional contrast injector 11 is integrated with the imaging device controller 10 (e.g., via a wired or wireless data connection), and the contrast injector 11 is controlled via the imaging device controller 10, including displaying the contrast injector settings in a (optionally selectable) window on the display of the imaging device controller 10.

As diagrammatically shown in FIG. 1, in some embodiments, a camera 16 (e.g., a video camera) is arranged to acquire a video stream or feed 17 of a portion of a workspace of the medical imaging device bay 3 that includes at least the area of the imaging device 2 where the local operator LO interacts with the patient, and optionally may further include the imaging device controller 10. In some embodiments, a microphone 15 is arranged to acquire an audio stream or feed 18 of the workspace so that the remote expert has auditory situational awareness as well. The video stream 17 and/or the audio stream 18 is sent to the assistance electronic device 12 via the communication link 14, e.g. as a streaming video feed received via a secure Internet link.

The communication link 14 also provides a natural language communication pathway 19 for verbal and/or textual communication between the local operator and the remote operator. For example, the natural language communication link 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14 providing the data communications 17, 18, e.g. the natural language communication pathway 19 may be provided via a landline telephone. In some embodiments, the natural language communication link 19 allows a local operator LO to call a selected remote expert RE. The call, as used herein, can refer to an audio call (e.g., a telephone call), a video call (e.g., a Skype or Facetime or other screen-sharing program), or an audio-video call. In another example, the natural language communication pathway 19 may be provided via a local electronic processing device, for example comprising an ROCC device 8, such as a mobile device (e.g., a tablet computer or a smartphone), or can be a wearable device worn by the local operator LO, such as an augmented reality (AR) display device (e.g., AR goggles), a projector device, a heads-up display (HUD) device, etc., each of which having a display device 36. For example, an “app” can run on the ROCC device 8 (operable by the local operator LO) and the assistance electronic device 12 (operable by the remote expert RE) to allow communication (e.g., audio chats, video chats, and so forth) between the local operator and the remote expert.

FIG. 1 also shows, in the remote service center 4 including the assistance electronic device 12, such as an electronic processing device, a workstation computer, or more generally a computer, which is operatively connected to receive and present the video feed 17 of the medical imaging device bay 3 from the camera 16 and/or to the audio feed 18. Additionally or alternatively, the assistance electronic device 12 can be embodied as a server computer or a plurality of server computers, e.g. interconnected to form a server cluster, cloud computing resource, or so forth. The workstation 12 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, and/or so forth). In some embodiments, the display device 24 can be a separate component from the workstation 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the workstation 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24. The video feed 17 from the camera 16 can also be displayed on the display device 24, and the audio feed 18 can be output on the assistance electronic device 12 via a loudspeaker 29. In some examples, the audio feed 18 can be an audio component of an audio/video feed (such as, for example, recording as a video cassette recorder (VCR) device would operate).

The medical imaging device controller 10 in the medical imaging device bay 3 also includes similar components as the assistance electronic device 12 disposed in the remote service center 4. Except as otherwise indicated herein, features of the medical imaging device controller 10, which includes a local workstation 12′, disposed in the medical imaging device bay 3 similar to those of the assistance electronic device 12 disposed in the remote service center 4 have a common reference number followed by a “prime” symbol, and the description of the components of the medical imaging device controller 10 will not be repeated. In particular, the medical imaging device controller 10 is configured to display a GUI 28′ on a display device or controller display 24′ that presents information pertaining to the control of the medical imaging device 2, such as configuration displays for adjusting configuration settings an alert 30 perceptible at the remote location when the status information on the medical imaging examination satisfies an alert criterion of the imaging device 2, imaging acquisition monitoring information, presentation of acquired medical images, and so forth. It will be appreciated that the screen mirroring data stream 18 carries the content presented on the display device 24′ of the medical imaging device controller 10. The communication link 14 allows for screen sharing between the display device 24 in the remote service center 4 and the display device 24′ in the medical imaging device bay 3. The GUI 28′ includes one or more dialog screens, including, for example, an examination/scan selection dialog screen, a scan settings dialog screen, an acquisition monitoring dialog screen, among others. The GUI 28′ can be included in the video feed 17 and displayed on the assistance electronic device display 24 at the remote location 4.

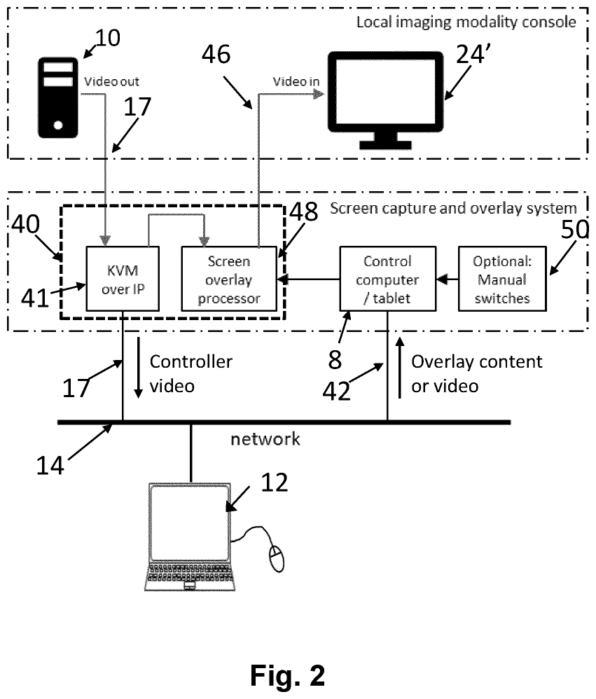

With reference to FIG. 2, and with continuing reference to FIG. 1, the apparatus 1 includes a video router 40 (diagrammatically shown as a rectangle in FIG. 1). In some examples, the video router 40 comprises a keyboard, video, monitor (KVM) switch 41. In other examples, the video router 40 may be constructed of a set of discrete video signal splitter and combiner components. The video router 40 is configured to receive a controller video 17 from the electronic imaging device controller 10, and also receive an overlay content or video 42 from the assistor electronic device 12. The controller video 17 is output to the assistor electronic device 12, where it is displayed on the display device 24 via the GUI 28 to allow the assistor to annotate the controller video displayed on the assistor electronic device 12.

The assistor electronic device 12 used by the remote expert RE is then programmed to provide inputs via the at least one user input device 22 indicative of graphical annotations 44 of the controller video 17. The graphical annotations 44 can include, for example, one or more of vectorized or symbolic graphical elements, bitmapped graphical elements, color elements, or transparency values. From this, the assistor electronic device 12 is configured to generate overlay content or video 42 that is, or is used to generate, annotated video 46 comprising at least the graphical annotations 44 to the controller display 24′ of the electronic imaging device controller 10. The annotated video 46 thus comprises the controller video 17 overlaid with the graphical annotations 44.

In one embodiment, the overlay content or video 42 is generated at the assistor electronic device 12 as the annotated video 46 comprising the controller video 17 overlaid with the graphical annotations 44, and the video router 40 is configured to output the annotated video 46 received from the assistor electronic device 12 to the controller display 24′. That is, the operation of combining the controller video 17 and the graphical annotations 44 is in this embodiment performed at the assistor electronic device 12 located with the remote expert RE.

In another embodiment, the overlay content or video 42 is generated as the graphical annotations 44 without the controller video 17, and the video router 40 includes a digital signal processor (DSP) 48 (identified as a screen overlay processor in FIG. 2) programmed to generate the annotated video 46 by overlaying the graphical annotations 44 received from the assistor electronic device 12 onto the controller video 17. That is, the operation of combining the controller video 17 and the graphical annotations 44 is in this embodiment performed at the video router 40 which is located with the local operator LO. In this embodiment, the overlay content or video 42 received at the video router 40 represents the graphical annotations 44. In one implementation, the overlay content or video 42 transmitted from the assistor electronic device 12 to the video router 40 is overlay content comprising the graphical annotations 44 represented as vectorized or symbolic graphical elements, e.g. represented as lines or shapes delineated by coordinates, along with color and transparency values. In this implementation, the DSP 48 of the video router 40 then converts these received vectorized or symbolic graphical elements to a screen overlay that is then fused with the controller video 17 to produce the annotated video 46. In another implementation, the overlay content or video 42 transmitted from the assistor electronic device 12 to the video router 40 comprises overlay video constructed as the graphical annotations 44 converted to bitmapped video containing only the graphical annotations. In this case the DSP 48 of the video router 40 then fuses the overlay video (which here contains only the annotations 44) and the controller video 17 to produce the annotated video 46. In either of these two implementations, the kill switch 50 can be implemented at the video router 40 by ceasing to fuse the received overlay content or video 42 with the controller video 17.

In the illustrative example of FIG. 2, the video router 40 is configured to receive the overlay content or video 42 via the assistor electronic device 12 via the ROCC device 8. However, in other embodiments the video router 40 may receive the overlay content or video 42 directly from the network 14, without the ROCC device 8 serving as a relay.

In some embodiments, the video router 40 also includes a manual overlay disable switch 50 operable by the local operator LO to switch the video router 40 from outputting the annotated video 46 to the controller display 24′ to outputting the controller video 17 to the controller display 24′. In variant embodiments in which the controller video 17 and the graphical annotations 44 are combined at the assistor electronic device 12 located with the remote expert RE, implementation of the kill switch 50 would entail sending the kill signal to the assistor electronic device 12, and the assistor electronic device 12 would then not add the annotations 44 into the controller video 17 in response to activation of the kill switch 50.

Furthermore, as disclosed herein, the ROCC device 8, the assistor electronic device 12, and the video router 40 are configured to perform a method or process 100 for providing assistance during a medical imaging examination performed using a medical imaging device 2 (i.e., by assisting local operators LO of respective medical imaging devices 2 during medical imaging examinations by a remote expert RE). The instructions to perform the method 100 are stored in the non-transitory computer readable medium 26 of the assistance electronic device 12, in the video router 40, and in the ROCC device 8.

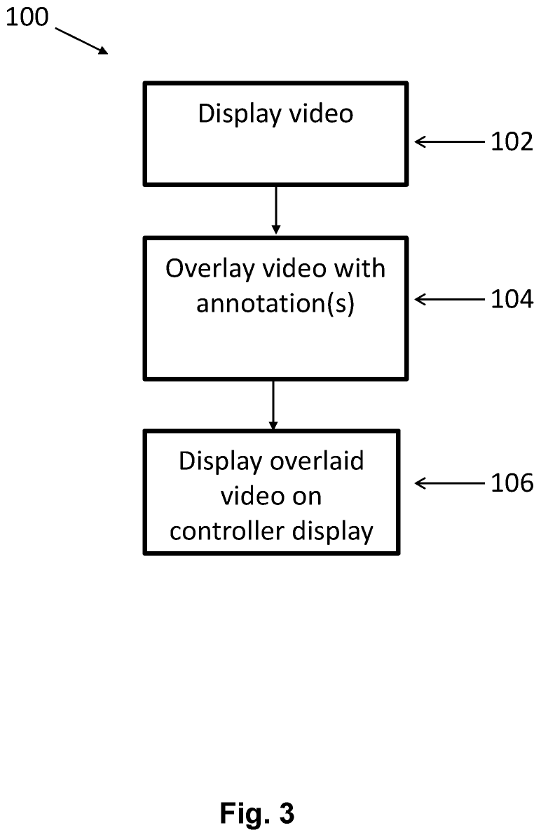

With reference to FIG. 3, and with continuing reference to FIGS. 1 and 2, an illustrative embodiment of the method 100 is diagrammatically shown as a flowchart. To begin the method 100, an imaging examination is commenced by the local operator LO using the medical imaging device 2. An event can occur during the examination which requires assistance from a remote expert RE. The controller video feed (acquired by the one or more cameras 16) is routed to the video router 40.

At an operation 102, the GUI 28 is provided on the assistor electronic device 12, and the controller video 17 is displayed thereon. At an operation 104, one or more graphical annotations 44 are received by the assistor electronic device 12 (i.e., input by the remote expert RE via the at least one user input device 22) and overlaid onto the controller video 17 to generate the annotated video 46. In some embodiments, the controller video 17 overlaid with the graphical annotations 44 is generated by a screen overlay component 48 that is part of the video router 40 and comprises a digital signal processing (DSP) component.

At an operation 106, the annotated video 46 is displayed on the controller display 24′ of the medical imaging device controller 10. In some embodiments, the controller video 17 is split i.e., via the KVM switch 41, or using a discrete video splitter) to split the controller video 17 into a plurality of feeds. One of the feeds is transmitted to the assistor electronic device 12, Another one of the feeds is transmitted to the screen overlay processor 48 which also receives the graphical annotations 44 from the assistor electronic device 12, that generates the controller video 17 overlaid with the graphical annotations 44 to generate the annotated video 46.

In another embodiment, the controller video 17 is an analog video, and the controller video 17 is converted to digital controller video that is processed by the DSP component 48 using an analog-to-digital converter (ADC) 52 implemented in the video router 40. The controller video 17 overlaid with the graphical annotations 44 (i.e., the annotated video 46) is converted to an analog signal via a digital-to-analog converter (DAC) 54 implemented in the video router 40.

In another embodiment, the displaying of the controller video 17 overlaid with the graphical annotations 44 on the controller display 24′ is switched to displaying the controller video 17 without the graphical annotations 44 on the controller display 24 in response to activation of the overlay disable switch 50 by the local operator LO.

In another embodiment, the annotated video 46 is displayed on the display device 36 of the ROCC device 8, and the graphical annotation(s) 44 are received from the local operator LO, in which the annotated video 46 includes the graphical annotation(s) 44 received from the local operator LO.

The system may offer the local operator LO a kill switch 50 to switch screen overlays on and off. This kill switch 50 can be realized by a touch or click button or switch displayed on a separate screen, or by a physical manual button or switch 50. The kill switch 50 enables the local operator LO to avoid distraction by stopping the process of overlaying the annotations 44 on the controller video 17.

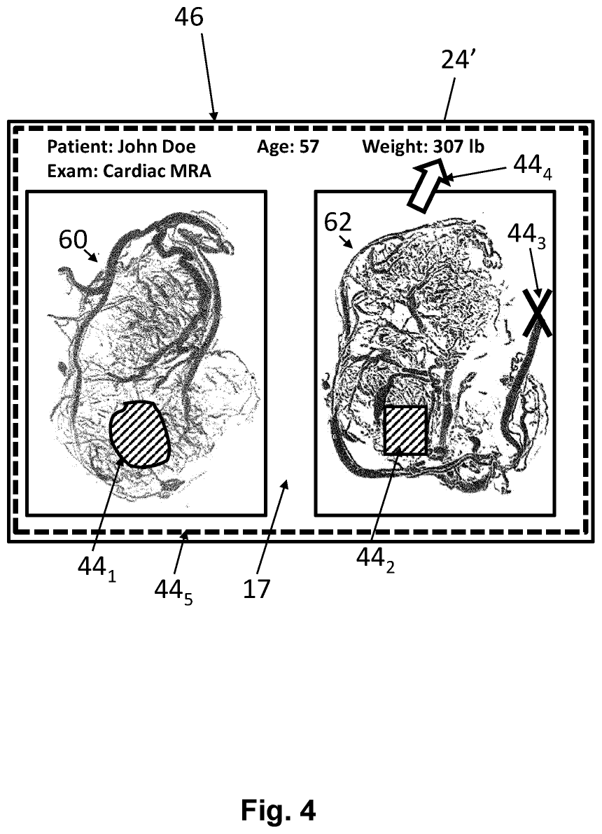

With reference to FIG. 4, some examples of highlighting overlay elements drawn by the remote expert RE are diagrammatically shown. FIG. 4 shows magnetic resonance angiography (MRA) images 60 and 62 such as might be displayed on the controller display 24′ during an MRA imaging session. It is to be appreciated that this is merely a nonlimiting illustrative example, and the disclosed approaches for remote expert annotation can be more generally employed in any medical imaging context (MR, CT, PET, et cetera). The display can also include text fields, such as the nonlimiting illustrative examples of “Patient: John Doe,” “Age: 57”, “Weight: 307 lb.”, and “Exam: Cardiac MRA”. FIG. 4 shows an example of the annotated video 46 including the combination of the controller video 17 and the graphical annotations 44₁, 44₂, 44₃, 44₄, and 44₅ drawn by the remote expert RE using the assistor electronic device 12. The overlay elements 44₁ and 44₂ are areas highlighted by the remote expert RE, while the annotation 44₃ is a location marker shown in FIG. 4 as an “X”, although other types of location markers are contemplated. The area overlay elements 44₁ and 44₂ can, for example, be hand drawn by the remote expert RE using a mouse pointer, touch-sensitive display, or the like. The annotation 44₄ is a superimposed graphical annotation cursor 44₄ operable by the remote expert RE to point out objects. In the illustrative example, the remote expert RE is using the superimposed graphical annotation cursor 44₄ to highlight the text “Weight: 307 lb.”, possibly as part of explaining that the imaging subject's weight may explain some aspect of the imaging examination. As can be seen, the various graphical annotations 44₁, 44₂, 44₃, and 44₄ drawn by the remote expert RE enable the remote expert to identify features of images, text, or other content shown on the controller display 24′, thus assisting in conveying information to the local operator LO. Overlay elements are preferably displayed in a transparent way so that information on the ROCC device 8 below the overlay elements is still visible to the local operator LO. Some examples of functional overlays shown in FIG. 4 include a border annotation 44₅ to indicates that this ROCC device 8 is being watched by the remote expert RE, and the pointer symbol 44₄ already described that mimics the mouse movement of the remote expert RE on the shared screen. The border annotation 44₅ is diagrammatically shown in FIG. 4 as a dashed line, but could be implemented for example as a red border to indicate the RE is observing.

The remote expert RE, viewing the medical imaging device controller 10 via the KVM 41, will need to interact with the video feed 17 to define screen overlay elements. For this reason, the assistance electronic device 12 includes a drawing tool for screen overlay elements. The drawing tool allows the remote expert RE to draw overlay elements directly onto the image of the shared screen.

The software then transforms the screen coordinates of the elements to the screen coordinates of the medical imaging device controller 10 (in case the screen resolutions or aspect ratios differ). Alternatively, the software calculates relative coordinates with respect to the screen extensions and leaves the determination of absolute coordinates to the ROCC device 8 or the screen overlay processor 48.

All information required for generating the overlays is transmitted through the network connection 14 to the ROCC device 8 in real time. This information includes, but is not limited to, coordinates, shape, color, and transparency level of all overlay elements; coordinates, shape, color, and transparency level of pointer, status information for additional functional overlays, such as activity of screen sharing, and so forth.

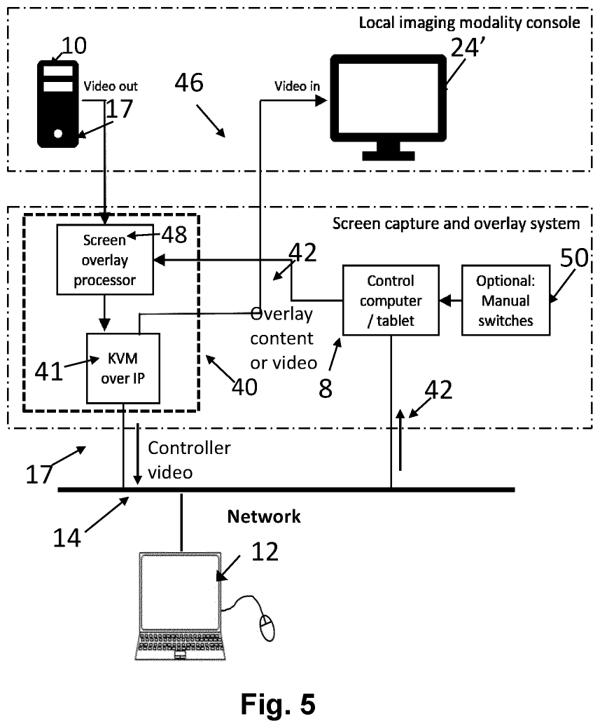

With reference to FIGS. 5-7, some alternative embodiments to the embodiment shown in FIG. 2 are presented. Each of these embodiments includes components analogous to those of FIG. 2 which are indicated by like reference numbers. As with the embodiment of FIG. 2, each of the embodiments of FIGS. 5-7 includes a video router 40 that receives controller video 17 from the electronic imaging device controller 10 and overlay content or video 42 from the assistor electronic device 12, and the video router 40 outputs the controller video 17 to the assistor electronic device 12, and also outputs the annotated video 46 to the controller display 24′, whereby the annotated video 46 is displayed on the controller display 24′.

In the embodiment of FIG. 5, the overlay content or video 42 is constructed before transmission via the KVM 41. This has the advantage that the remote operator using the assistor electronic device 12 sees exactly what the local operator sees on the controller display 24′. One possible disadvantage of the approach of FIG. 5 is that drawing elements may seem to have to some delay due to network latency.

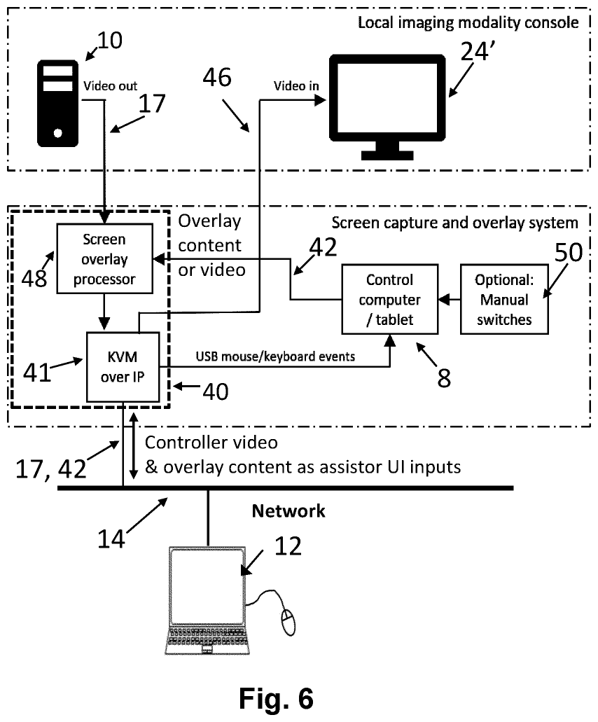

The embodiment of FIG. 6 is similar to that of FIG. 5, but in the embodiment of FIG. 6 the overlay content or video 42 is in the form of overlay content 42 represented by UI inputs at the assistor electronic device 12 (e.g., keyboard and mouse inputs 42) made by the operator of the assistor electronic device 12 in drawing, typing, or otherwise creating the graphical annotations 44. These UI inputs forming the overlay content 42 are transmitted through the KVM 41 and fed to the ROCC device 8 which generates the overlay content or video 42 that is then input to the video router 40.

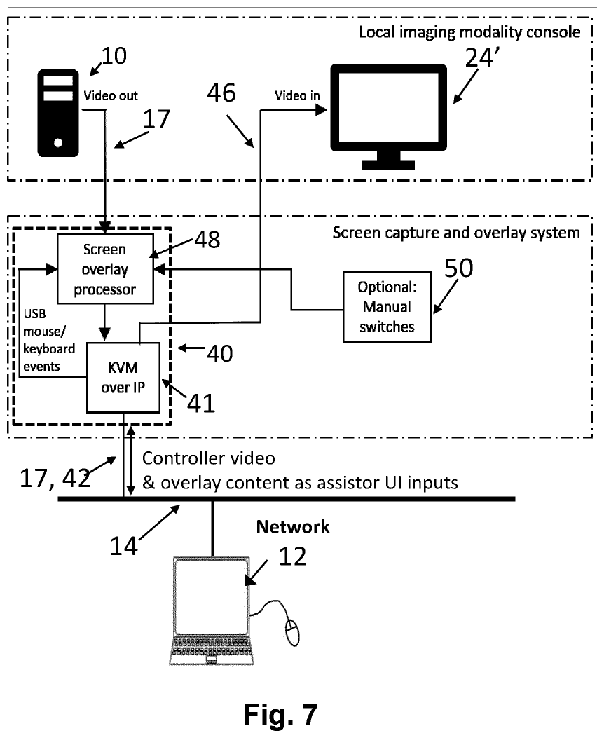

In the embodiment of FIG. 7, a similar system to that of FIG. 6 is shown, except that in FIG. 7 the UI inputs forming the overlay content 42 are input directly to the screen overlay processor 48 (rather than to the ROCC device 8), and the screen overlay processor 48 interprets the UI inputs forming the overlay content 42 directly to produce the annotated video 46 which is then fed to the controller display 24′ via the KVM 41.

In the following, some further variant embodiments are described.

In some embodiments, the graphical overlay elements have additional parameters controlling animations, flashing, etc. to increase visibility.

In some embodiments, the remote expert RE or the ROCC device 8 must request the sharing of overlays. The local operator LO must then acknowledge the request before the screen overlay functionality is activated.

In some embodiments, the local operator LO can interact with the overlay elements on the ROCC device 8 using the local pointer device (mouse etc.). To enable this, the movements of the local pointing device must be processed by the ROCC device 8 or the screen overlay processor 48. The KVM 41, being connected not only to the annotation(s) 44, but also to the pointing device, can facilitate this functionality.

Aspects of the invention may be implemented in a computer program product, which may be a collection of computer program instructions stored on a computer readable storage device which may be executed by a computer. The instructions of the present invention may be in any interpretable or executable code mechanism, including but not limited to scripts, interpretable programs, dynamic link libraries (DLLs) or Java classes. The instructions can be provided as complete executable programs, partial executable programs, as modifications to existing programs (e.g. updates) or extensions for existing programs (e.g. plugins). Moreover, parts of the processing of the present invention may be distributed over multiple computers or processors.

A processor or processing unit) may comprise or consist of a controller for implementing the control method. The controller can be implemented in numerous ways, with software and/or hardware, to perform the various functions required. A processor is one example of a controller which employs one or more microprocessors that may be programmed using software (e.g., microcode) to perform the required functions. A controller may however be implemented with or without employing a processor, and also may be implemented as a combination of dedicated hardware to perform some functions and a processor (e.g., one or more programmed microprocessors and associated circuitry) to perform other functions.

Examples of controller components that may be employed in various embodiments of the present disclosure include, but are not limited to, conventional microprocessors, application specific integrated circuits (ASICs), and field-programmable gate arrays (FPGAs).

In various implementations, a processor or controller may be associated with one or more storage media such as volatile (transitory) and non-volatile (non-transitory) computer memory such as RAM, PROM, EPROM, and EEPROM. The storage media may be encoded with one or more programs that, when executed on one or more processors and/or controllers, perform at the required functions. Various storage media may be fixed within a processor or controller, may be transportable or may be available on-demand (e.g., via the cloud), such that the one or more programs stored thereon can be loaded into a processor or controller. While the invention has been illustrated and described in detail in the drawings and foregoing description, such illustration and description are to be considered illustrative or exemplary and not restrictive; the invention is not limited to the disclosed embodiments.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. An assistive system for providing remote assistance to a local operator of a medical imaging device having an electronic imaging device controller and a controller display, the assistive system comprising:
an assistor electronic device including an processor, an assistor display, and at least one user input device;
a video router configured to:
receive controller video from the electronic imaging device controller;
receive overlay content or video from the assistor electronic device;
output the controller video to the assistor electronic device; and
output annotated video to the controller display, whereby the annotated video is displayed on the controller display;
wherein the assistor electronic device is programmed to:
provide a graphical user interface operative to display the controller video on the assistor display; and
receive graphical annotations of the controller video from the at least one user input device, the assistor electronic device further programmed to generate the overlay content or video comprising at least the graphical annotations; and
wherein the annotated video comprises the controller video overlaid by the video router, with the graphical annotations.

2. The assistive system of claim 1, wherein the assistor electronic device is programmed to generate the overlay content or video as the annotated video comprising the controller video overlaid with the graphical annotations, and the video router is configured to output the annotated video received from the assistor electronic device to the controller display.

3. The assistive system of claim 1, wherein the assistor electronic device is programmed to generate the overlay content or video comprising the graphical annotations without the controller video, and the video router includes a digital signal processor programmed to generate the annotated video by combining the overlay content or video received from the assistor electronic device with the controller video.

4. The assistive system of claim 3, wherein the overlay content or video received from the assistor electronic device is overlay content in which the graphical annotations are represented as vectorized or symbolic graphical elements, and the digital signal processor is programmed to generate the annotated video by converting the vectorized or symbolic graphical elements to a screen overlay and fusing the screen overlay with the controller video to produce the annotated video.

5. The assistive system of claim 3, wherein the overlay content or video received from the assistor electronic device is overlay video in which the graphical annotations are represented as overlay video, and the digital signal processor (48) is programmed to generate the annotated video by fusing the overlay video with the controller video to produce the annotated video.

6. The assistive system of claim 4, wherein the video router includes an overlay disable switch operable by the local operator to cause the video router to stop combining the overlay content or video received from the assistor electronic device with the controller video.

7. The assistive system of claim 1, further comprising:
a local electronic device operable by the local operator, wherein the video router is configured to receive the overlay content or video from the assistor electronic device via the local electronic device and to output the controller video to the assistor electronic device via the local electronic device.

8. The assistive system of claim 1, wherein the video router comprises a keyboard, video, monitor switch.

9. A method of providing assistance from a remote expert to a local operator of a medical imaging device during a medical imaging examination, the method comprising:
receiving controller video output between an electronic controller of the medical imaging device and an assister electronic device;
providing a user interface on an assistor electronic device operable by the remote expert, the UI displaying controller video;
receiving, via at least one user input device of the assistor electronic device, overlay content;
combining the overlay content with the controller video between the electronic controller and the assister electronic device; and
displaying the controller video overlaid with the graphical annotations on a controller display of the medical imaging device.

10. The method of claim 9, further including:
splitting the controller video into a plurality of feeds;
transmitting one of the feeds to the assistor electronic device; and
transmitting another of the feeds to a screen overlay processor that also receives the graphical annotations from the assistor electronic device and that generates the controller video overlaid with the graphical annotations.

11. The method of claim 10, wherein the splitting comprises employing a Keyboard, Video, & Mouse switch to split the feed into the plurality of feeds.

12. The method of claim 9, wherein the assistor electronic device implements a screen overlay component configured to generate the controller video overlaid with the graphical annotations.

13. The method of claim 9, wherein the controller video overlaid with the graphical annotations is generated by a screen overlay component that comprises a digital signal processing component.

14. The method of claim 13, wherein the controller video is an analog signal, the method further comprising:
converting the controller video to digital controller video that is processed by the DSP component using an analog-to-digital converter; and
converting the controller video overlaid with the graphical annotations to an analog signal that is displayed by the controller display using a digital-to-analog converter.

15. The method of claim 9, wherein the graphical annotations comprise one or more of vectorized or symbolic graphical elements, color elements, or transparency values.

16. The method of claim 9, further comprising:
switching from displaying the controller video overlaid with the graphical annotations on the controller display to displaying the controller video without the graphical annotations on the controller display in response to activation of an overlay disable switch by the local operator.

17. The method of claim 9, wherein the controller display includes a touchscreen, and the method further includes:
receiving, via the touchscreen, one or more graphical annotations from the local operator, wherein the controller video is further overlaid with the graphical annotations from the local operator.

18. An assistive system for providing remote assistance to a local operator of a medical imaging device having an electronic imaging device controller and a controller display, the assistive system comprising:
an assistor electronic device including an processor, an assistor display, and at least one user input device;
a video router configured to:
receive controller video from the electronic imaging device controller;
receive graphical annotations from the assistor electronic device;
output the controller video to the assistor electronic device; and
output annotated video to the controller display, whereby the annotated video is displayed on the controller display;
wherein the assistor electronic device is programmed to:
provide a graphical user interface operative to display the controller video on the assistor display; and
receive the graphical annotations of the controller video from the at least one user input device; and
wherein the annotated video comprises the controller video overlaid by the video router, with the graphical annotations.

19. The assistive system of claim 18, wherein the assistor electronic device is programmed to represent the graphical annotations as vectorized or symbolic graphical elements, and the video router includes a digital signal processor programmed to generate the annotated video by combining the vectorized or symbolic graphical elements received from the assistor electronic device with the controller video.

20. The assistive system of claim 18, wherein the video router includes an overlay disable switch operable by the local operator to stop the overlaying of the controller video with the graphical annotations.

21. The assistive system of claim 18, further comprising:
a local electronic device operable by the local operator, wherein the video router is configured to receive the graphical annotations from the assistor electronic device via the local electronic device and to output the controller video to the assistor electronic device via the local electronic device.
